(19) United States
(12) Patent Application Publication
(10) Pub. No.: US 2022/0341925 A1
(43) Pub. Date: Oct. 27, 2022

(54) Digital Microfluidic Chemiluminescence Detection Chip, Detection Method and Detection Device

(71) Applicants: Beijing BOE Sensor Technology Co., Ltd., Beijing (CN); BOE Technology Group Co., Ltd., Beijing (CN)

(72) Inventors: Yue LI, Beijing (CN); Wenliang YAO, Beijing (CN); Nan ZHAO, Beijing (CN); Yongjia GAO, Beijing (CN); Bolin FAN, Beijing (CN); Le GU, Beijing (CN); Hui LIAO, Beijing (CN); Yingying ZHAO, Beijing (CN)

(21) Appl. No.: 17/434,408

(22) PCT Filed: Feb. 7, 2021
(86) PCT No.: PCT/CN2021/075848

§ 371 Date: Aug. 26, 2021

(30) Foreign Application Priority Data
Feb. 25, 2020 (CN) 202010114438.8

(51) Int. Cl. G01N 33/543; G01N 21/76

(52) U.S. Cl. CPC G01N 33/54386; G01N 33/54326; G01N 21/76

(57) ABSTRACT

The present disclosure relates to a digital microfluidic chemiluminescence detection chip, a detection method and a detection device. The digital microfluidic chemiluminescence detection chip includes a first baseplate and a second baseplate disposed oppositely. A cavity formed by the first and second baseplate includes a mixing and incubating area for combining an antigen, a magnetic particle antibody and an antibody, a luminescence detection area for chemiluminescence and detecting an optical signal, and a communication path for communicating the mixing and incubating area and the luminescence detection area. The first baseplate is provided with a drive array for driving sample solution to move and an optical sensing array for acquiring a luminescence signal of the sample solution. The drive array corresponds to positions of the mixing and incubating area, the luminescence detection area and the communication path. The optical sensing array corresponds to a position of the luminescence detection area.

### CROSS-REFERENCE TO RELATED APPLICATIONS

The present disclosure is a U.S. National Phase Entry of International Application PCT/CN2021/075848 having an international filing date of Feb. 7, 2021, which claims priority of Chinese patent application No. 202010114438.8 filed to CNIPA on Feb. 25, 2020, and titled “Digital Microfluidic Chemiluminescence Detection Chip, Detection Method and Detection Device”, the contents of which should be construed as being hereby incorporated by reference in their entirety.

### TECHNICAL FIELD

The embodiments of the present disclosure relates to, but are not limited to, the technical field of chemiluminescence detection, in particular to a digital microfluidic chemiluminescence detection chip, a detection method and a detection device.

### BACKGROUND

Chemiluminescence analysis is an analytical method to determine the content of substances according to intensity of radiation light generated by chemical reaction. Chemiluminescence immunoassay realizes quantitative and qualitative detection of antigens or antibodies by combining chemiluminescence analysis with immune reaction analysis, labeling antibodies or antigens with chemiluminescence related substances, separating free chemiluminescence labels after reaction with antigens or antibodies to be detected, and adding other related substances of a chemiluminescence system to produce chemiluminescence.

The technology of chemiluminescence immunoassay has high accuracy and specificity, has become one of the most important technologies in test methodology, and is commonly recognized as one of the advanced labeled immunoassay technologies in the world. As the main means of disease diagnosis, the chemiluminescence immunoassay has been widely applied in in-vitro diagnosis and detection of body immunity functions, infectious diseases, endocrine systems, tumor marks, sex hormones, thyroid function and so on.

The existing chemiluminescence detection devices have defects of large system volume and low consistency of detection results.

### SUMMARY

The following is a summary of the subject matter detailed herein. This summary is not intended to limit the scope of protection of the claims.

An embodiment of the present disclosure provides a digital microfluidic chemiluminescence detection chip, including a first baseplate and a second baseplate disposed oppositely, wherein a cavity formed by the first baseplate and the second baseplate includes a mixing and incubating area configured to implement the combination of an antigen, a magnetic particle antibody and an antibody, a luminescence detection area configured to implement chemiluminescence and detecting an optical signal, and a communication path configured to communicate the mixing and incubating area with the luminescence detection area, the first baseplate is provided with a drive array configured to drive a sample solution to move and an optical sensing array configured to acquire a luminescence signal of the sample solution, the drive array corresponds to positions of the mixing and incubating area, the luminescence detection area and the communication path, and the optical sensing array corresponds to a position of the luminescence detection area.

In some possible embodiments, the mixing and incubating area includes a magnetic particle filling dish and a magnetic particle mixing channel. The magnetic particle filling dish is configured to provide the magnetic particle antibody to the magnetic particle mixing channel, in the magnetic particle mixing channel, the sample solution moves along the magnetic particle mixing channel under drive of the drive array to make the antigen in the sample solution be combined with the magnetic particle antibody to form a first incubation sample solution, and the first incubation sample solution includes an antigen-magnetic particle antibody complex.

In some possible embodiments, the mixing and incubating area further includes an enzyme label filling dish and an enzyme label mixing channel, the enzyme label filling dish is configured to provide an enzyme labeled antibody to the enzyme label mixing channel, the enzyme label mixing channel is communicated with the magnetic particle mixing channel, in the enzyme label mixing channel, the first incubation sample moves along the enzyme label mixing channel under the drive of the drive array to make the first incubation sample solution be combined with the enzyme labeled antibody to form a second incubation sample solution, and the second incubation sample solution includes an antigen-magnetic particle antibody-enzyme labeled antibody complex.

In some possible embodiments, the luminescence detection area includes a substrate filling dish and a purification channel, the purification channel is communicated with the mixing and incubating area, the substrate filling dish is configured to provide a luminescent substrate to the purification channel, in the purification channel, the second incubation sample moves along the purification channel under the drive of the drive array to make the second incubation sample be combined with the luminescent substrate to form a third incubation sample solution, and the third incubation sample solution includes an antigen-magnetic particle antibody-enzyme labeled antibody-luminescent substrate complex.

In some possible embodiments, the magnetic particle mixing channel, the enzyme label mixing channel and the purification channel are annular channels, and mixing of the sample solution and the magnetic particle antibody, mixing of the first incubation sample solution and the enzyme labeled antibody, and mixing of the second incubation sample solution and the luminescent substrate are implemented by circling.

In some possible embodiments, the luminescence detection area further includes a wash filling dish, the wash filling dish is configured to provide a wash buffer to the purification channel, and in the purification channel, the second incubation sample solution moves along the purification channel under the drive of the drive array to implement mixing of the second incubation sample solution and the wash buffer; an external magnetic control device fixes the magnetic particle antibody in the second incubation sample solution, and an impurity solution in the second incubation sample solution is discharged from the purification channel under the drive of the drive array.

In some possible embodiments, the luminescence detection area further includes a detection area, the detection area is communicated with the purification channel, and in the detection area, the optical sensing array acquires an optical signal of chemiluminescence of the third incubation sample solution and converts the optical signal into an electrical signal.

In some possible embodiments, the second baseplate is provided with multiple filling holes, and the multiple filling holes correspond to positions of the magnetic particle filling dish, the enzyme label filling dish, the wash filling dish and the substrate filling dish respectively.

In some possible embodiments, the digital microfluidic chemiluminescence detection chip further includes a filling area and a waste liquid area, the filling area is communicated with the mixing and incubating area and is configured to receive a sample solution to be detected, and the waste liquid area is communicated with the luminescence detection area and is configured to receive a waste liquid from the luminescence detection area.

In some possible embodiments, the drive array adopts an active drive implementation mode.

In some possible embodiments, the first baseplate includes a first base substrate, an array structure layer disposed on a side of the first base substrate facing the second baseplate and a first hydrophobic layer disposed on a side of the array structure layer facing the second baseplate, the second baseplate includes a second base substrate and a second hydrophobic layer disposed on a side of the second base substrate facing the first baseplate, the drive array and the optical sensing array are disposed in the array structure layer, the drive array includes multiple drive units, each drive unit includes a drive transistor and a drive electrode, the drive electrode is connected to the drive transistor, the optical sensing array includes multiple optical sensing units, each optical sensing unit includes a sensing transistor and a photodiode, and the photodiode is connected to the sensing transistor.

In some possible embodiments, the array structure layer includes:

a first base substrate;

a drive gate electrode and a sensing gate electrode disposed on the first base substrate;

a first insulating layer covering the drive gate electrode and the sensing gate electrode;

a drive active layer and a sensing active layer disposed on the first insulating layer;

a drive source electrode and a drive drain electrode with adjacent ends respectively disposed on the drive active layer, and a sensing source electrode and a sensing drain electrode with adjacent ends disposed on the sensing active layer;

a second insulating layer and a third insulating layer covering the drive source electrode, the drive drain electrode, the sensing source electrode and the sensing drain electrode, and provided with a first hole exposing the sensing drain electrode;

a photodiode disposed on the third insulating layer, wherein a first electrode of the photodiode is connected with the sensing drain electrode through the first hole;

a fourth insulating layer covering the photodiode and provided with a second hole exposing the drive drain electrode;

a drive electrode disposed on the fourth insulating layer, wherein the drive electrode is connected with the drive drain electrode through the second hole; and

a fifth insulating layer covering the drive electrode.

An embodiment of the present disclosure further provides a digital microfluidic chemiluminescence detection device, including the digital microfluidic chemiluminescence detection chip, and further including a solution transfer device, a temperature control device, a magnetic control device and a signal processing device, wherein the solution transfer device is configured to transfer a sample solution onto the digital microfluidic chemiluminescence detection chip, the temperature control device is configured to provide a set temperature to the digital microfluidic chemiluminescence detection chip, the magnetic control device is configured to provide a set magnetic field to the digital microfluidic chemiluminescence detection chip, and the signal processing device is connected with the digital microfluidic chemiluminescence detection chip and is configured to read an electrical signal of the optical sensing array, and analyze and process the electrical signal to obtain concentration information.

In some possible embodiments, the temperature control device is disposed on a side of the first baseplate away from the second baseplate or a side of the second baseplate away from the first baseplate and is configured to provide the set temperature to the mixing and incubating area; the magnetic control device is disposed on the side of the first baseplate away from the second baseplate or the side of the second baseplate away from the first baseplate and is configured to provide the set magnetic field to the luminescence detection area.

An embodiment of the present disclosure further provides method for detecting digital microfluidic chemiluminescence using the digital microfluidic chemiluminescence detection chip, which includes:

driving, by the drive array, a sample solution to be sequentially combined with the magnetic particle antibody, the enzyme labeled antibody and the luminescent substrate to form an antigen-magnetic particle antibody-enzyme labeled antibody-luminescent substrate complex; and

acquiring, by the optical sensing array, an optical signal of chemiluminescence of the antigen-magnetic particle antibody-enzyme labeled antibody-luminescent substrate complex, and converting the optical signal into an electrical signal.

In some possible embodiments, driving, by the drive array, the sample solution to be sequentially combined with the magnetic particle antibody, the enzyme labeled antibody and the luminescent substrate to form the antigen-magnetic particle antibody-enzyme labeled antibody-luminescent substrate complex includes:

driving, by the drive array, the sample solution to be sequentially combined with the magnetic particle antibody and the enzyme labeled antibody in the mixing and incubating area to form an antigen-magnetic particle antibody-enzyme labeled antibody complex; and

driving, by the drive array, the antigen-magnetic particle antibody-enzyme labeled antibody complex to be combined with the luminescent substrate in the luminescence detection area to form the antigen-magnetic particle antibody-enzyme labeled antibody-luminescent substrate complex.

Of course, the implementation of any product or method of the present disclosure does not necessarily need to realize all the advantages mentioned above at the same time. Other features and advantages of the present disclosure will be described in subsequent embodiments in the description, and, in part, become apparent from the embodiments in the description, or can be understood by implementing the embodiments of the present disclosure. The purpose and other advantages of the embodiments of the present disclosure may be realized and obtained through the structure specifically pointed out in the description, the claims and the drawings.

Other aspects can be understood upon reading and understanding of the drawings and the detailed description.

### BRIEF DESCRIPTION OF DRAWINGS

The drawings are used to provide a further understanding of technical solutions of the present disclosure and constitute a part of the description, which are used together with the embodiments of the present disclosure to explain the technical solutions of the present disclosure and do not constitute limitations on the technical solutions of the present disclosure. The shape and size of the components in the drawings do not reflect the actual scale, and the purpose thereof is only to describe the contents of the present disclosure.

FIG. 1 illustrates a schematic diagram of a structure of a digital microfluidic chemiluminescence detection device according to an embodiment of the present disclosure.

FIG. 2 illustrates a schematic diagram of a structure of a digital microfluidic chemiluminescence detection chip according to an embodiment of the present disclosure.

FIG. 3 illustrates a schematic diagram of a structure of a digital microfluidic chemiluminescence detection chip according to an embodiment of the present disclosure.

FIG. 4 illustrates a schematic diagram of a sample solution incubation process according to an embodiment of the present disclosure.

FIG. 5 illustrates a schematic diagram of a structure of a digital microfluidic chemiluminescence detection device according to an embodiment of the present disclosure.

FIG. 6 illustrates a schematic diagram of a structure of a digital microfluidic chemiluminescence detection chip integrated with an optical sensing array according to an embodiment of the present disclosure.

FIG. 7 illustrates a schematic diagram of a structure of an array structure layer according to an embodiment of the present disclosure.

### DETAILED DESCRIPTION

The following embodiments are used to describe the present disclosure, but are not used to limit the scope of the present disclosure. It should be noted that the embodiments in the present disclosure and the features in the embodiments may be combined randomly with each other if there is no conflict.

In recent years, with the development of clinical laboratory medicine, miniaturized portable instant detection instruments have become one of the development trends of clinical diagnostic instruments. Point of Care Test (POCT), i.e., bedside test or near patient test, is a method for obtaining test results in several minutes by using a portable apparatus, is widely applicable to hospitals, nursing wards, rescue units, insurance companies and family health networks, and is also applicable to special environments, such as emergency relief, remote rural areas and ways of marching. The emergence of POCT allows that the work traditionally done by professional inspectors can be done by non-professional inspectors to a greater extent.

Miniaturized Total Analysis System (μ-TAS) was first proposed by Manz and Widmer of Ciba Geigy Company in 1990, and then it has been developed rapidly. Microfluidic chip is the main development direction and the most active frontier field of the miniaturized total analysis system. Its goal is to integrate functions of the whole laboratory, including sampling, dilution, reagent addition, reaction, separation and detection on a microchip. Compared with traditional biochemical analysis laboratory, the microfluidic chip has advantages such as automatic operation, fast detection, small volume and low sample consumption, which will bring about revolutionary scientific and technological changes in biochemical analysis and medical diagnosis. For the first developed channel type microfluidic chip, since it needs to achieve liquid drive control by peripheral micro pumps, micro valves and complex pipelines, bubbles and “dead zone effect” will easily exist in the channel. Once the channel is formed, it can only be used for specific applications, thus it lacks flexibility. These problems restrict the wide application of the channel type microfluidic chip. In 1993, Berge found the dielectric wetting phenomenon through experiments, and fully verified the principle and influencing factors of dielectric wetting in realizing droplet manipulation. Since then, the digital microfluidics technology has been developed vigorously. Digital microfluidic chip is based on the principle of dielectric wetting, and by changing the hydrophilicity and hydrophobicity of droplets, can apply driving force to discrete micro droplets to control their movement. It can control fluids at a micrometer scale, and it has the ability to miniaturize the basic functions of biological and chemical laboratories to a chip with a few square centimeters. Therefore, the digital microfluidic chip is also called Laboratory on a Chip (LOC), which has advantages such as small size, portability, flexible combination of functions and high integration level. Digital microfluidics is divided into active matrix digital microfluidics and passive matrix digital microfluidics. The main difference between them is that active matrix digital microfluidics drive droplets in an array mode, which can accurately control a droplet at a certain position to move separately, while passive digital microfluidics drive droplets at all positions to move or stop at the same time. In recent years, the digital microfluidic chip, as a new technology of micro liquid control, has shown great potential and application prospects in the fields of biology, chemistry, medicine, especially POCT, due to its advantages such as simple structure, small required amount of sample and reagent, easiness in integration, parallel processing and easiness in automation.

At present, related chemiluminescence detection devices usually adopt a structure with a liquid path system and an external light detection device. The liquid path system includes precision pumps such as a vacuum pump, a flushing pump, a matrix liquid pump and a peristaltic pump. The external light detection device includes a convex lens, a photomultiplier tube, a photocell, etc. The structure needs complex ancillary pipelines, pumps and peripheral optical paths, which not only increases the volume of the system, but also causes high signal-to-noise ratio and low consistency of detection results.

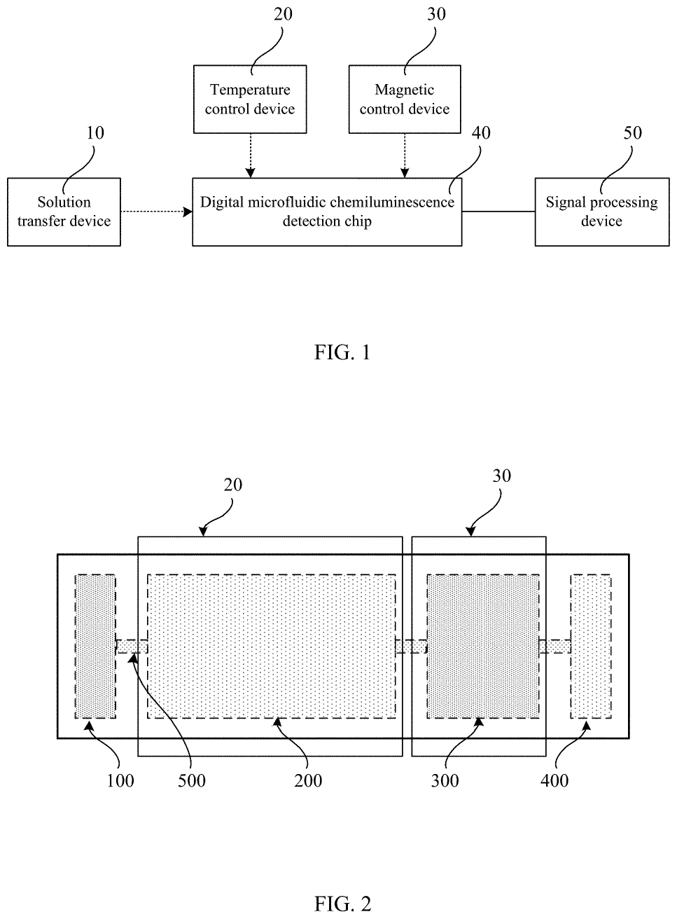

An embodiment of the present disclosure provides a digital microfluidic chemiluminescence detection device. FIG. 1 illustrates a schematic diagram of a structure of the digital microfluidic chemiluminescence detection device according to the embodiment of the present disclosure. As shown in FIG. 1, the digital microfluidic chemiluminescence detection device according to the embodiment of the present disclosure includes a solution transfer device 10, a temperature control device 20, a magnetic control device 30, a digital microfluidic chemiluminescence detection chip 40 integrated with an optical sensing array, and a signal processing device 50. The solution transfer device 10 is configured to transfer a sample solution onto the digital microfluidic chemiluminescence detection chip 40. The temperature control device 20 is configured to provide set temperature to the digital microfluidic chemiluminescence detection chip 40. The magnetic control device 30 is configured to provide a set magnetic field to the digital microfluidic chemiluminescence detection chip 40. The digital microfluidic chemiluminescence detection chip 40 is configured to combine an antigen, a magnetic particle antibody, an antibody and a luminescent substrate, acquire an optical signal of chemiluminescence by using the integrated optical sensing array and convert the optical signal into an electrical signal. The signal processing device 50 is connected with the digital microfluidic chemiluminescence detection chip 40 and is configured to read the electrical signal of the optical sensing array, and analyze and process the electrical signal to obtain concentration information.

The digital microfluidic chemiluminescence detection device provided by the embodiment of the present disclosure, achieves the preparation of the complex sample solution of chemiluminescence reaction by using the digital microfluidic technology to, avoids the complex fluid path system. By integrating the optical sensing array in the digital microfluidic chemiluminescence detection chip, acquisition of the optical signal in the chip and avoids the complex peripheral optical path structure. The embodiment of the present disclosure has features such as compact structure, small volume, low power consumption and low cost, reduces the signal-to-noise ratio of the signal and improves the consistency of detection results.

FIG. 2 illustrates a schematic diagram of a structure of a digital microfluidic chemiluminescence detection chip according to an embodiment of the present disclosure. As shown in FIG. 2, the digital microfluidic chemiluminescence detection chip 40 includes a cavity formed by a first baseplate and a second baseplate aligned with each other, wherein the cavity is divided into multiple functional areas, the multiple functional areas include a filling area 100, a mixing and incubating area 200, a luminescence detection area 300 and a waste liquid area 400, a communication path 500 is disposed between the multiple functional areas, the filling area 100 and the mixing and incubating area 200 are communicated through the communication path 500, the mixing and incubating area 200 and the luminescence detection area 300 are communicated through the communication path 500, and the luminescence detection area 300 and the waste liquid area 400 are communicated through the communication path 500. A drive array for driving the sample solution to move is disposed on the first baseplate where the multiple functional areas and the communication path are located, an optical sensing array for acquiring the luminescence signal of the sample solution is disposed on the first baseplate where the luminescence detection area 300 is located, and a hydrophobic layer is disposed on the surfaces of the drive array and the optical sensing array.

As shown in FIG. 2, the temperature control device 20 is disposed on the side of the first baseplate away from the second baseplate or on a side of the second baseplate away from the first baseplate, corresponds to the position of an area where the mixing and incubating area 200 is located, and is configured to provide set temperature to the mixing and incubating area 200. The magnetic control device 30 is disposed on the side of the first baseplate away from the second baseplate or the side of the second baseplate away from the first baseplate, corresponds to the position of an area where the luminescence detection area 300 is located, and is configured to provide a set magnetic field to the luminescence detection area 300. In an embodiment of the present disclosure, the temperature control device 20 may include a heater, a temperature sensor, a first controller and so on, such as a resistance wire or a semiconductor thermoelectric cooler. The heater, the temperature sensor and the first controller form a closed-loop control to accurately and effectively control the temperature of the mixing and incubating area 200. The reaction temperature of chemiluminescence immunoassay may be controlled at 37±0.5° C. The magnetic control device 30 may include a magnet (permanent magnet or electromagnet), a second controller and so on. The second controller controls intensity of the magnetic field provided to the luminescence detection area 300 by adjusting a distance between the permanent magnet and the first baseplate or the second baseplate or by switching on and off the electromagnet. In practical implementation, the temperature control device 20 and the magnetic control device 30 may be disposed separately or in combination to form a temperature control and magnetic control integrated device.

As shown in FIG. 2, the filling area 100 is configured to receive the sample solution to be detected transferred by the solution transfer device 10. The mixing and incubating area 200 is communicated with the filling area 100 through the communication path 500 and is configured to form a first incubation sample solution and a second incubation sample solution sequentially under the control of the drive array in the digital microfluidic chemiluminescence detection chip. The first incubation sample solution is an antigen-magnetic particle antibody complex. The second incubation sample solution is an antigen-magnetic particle antibody-enzyme labeled antibody complex. The luminescence detection area 300 is communicated with the mixing and incubating area 200 through the communication path 500 and is configured to wash the second incubation sample solution and form a third incubation sample solution sequentially under the control of the magnetic control device 30 and the drive array, and acquire an optical signal of chemiluminescence of the third incubation sample solution by using the optical sensing unit of the digital microfluidic chemiluminescence detection chip. The third incubation sample solution is an antigen-magnetic particle antibody-enzyme labeled antibody-luminescent substrate complex. The waste liquid area 400 is communicated with the luminescence detection area 300 through the communication path 500 and is configured to store the waste liquid from the luminescence detection area 300.

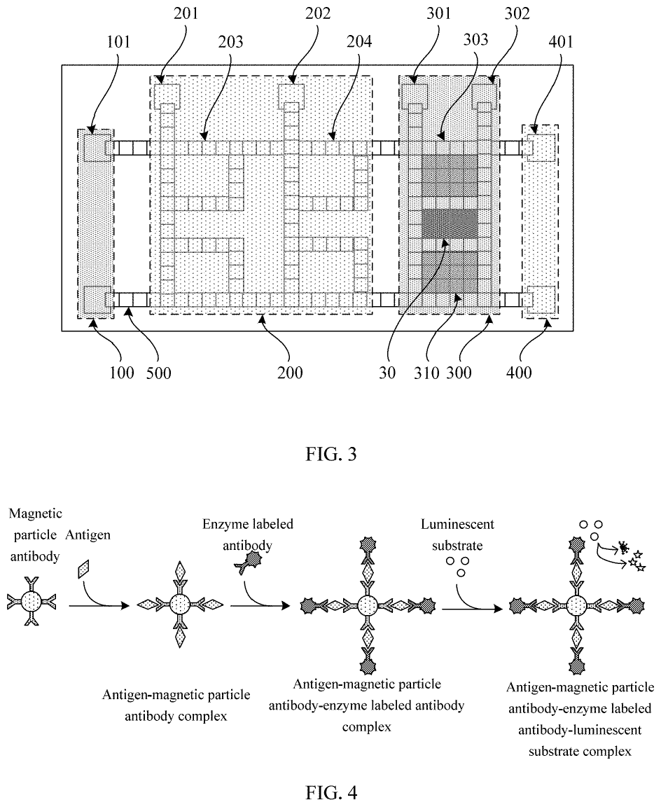

FIG. 3 is a schematic diagram of a structure of a digital microfluidic chemiluminescence detection chip according to an embodiment of the present disclosure, which illustrates a dual-channel structure that can implement simultaneous detection of two sample solutions. As shown in FIG. 3, in a plane parallel to the chip, the digital microfluidic chemiluminescence detection chip 40 includes multiple functional areas, which are respectively a filling area 100, a mixing and incubating area 200, a luminescence detection area 300 and a waste liquid area 400. The filling area 100 and mixing and incubating area 200 are communicated through a communication path 500. The mixing and incubating area 200 and the luminescence detection area 300 are communicated through the communication path 500. The luminescence detection area 300 and the waste liquid area 400 are communicated through the communication path 500. In a plane perpendicular to the chip, the digital microfluidic chemiluminescence detection chip 40 includes a first baseplate and a second baseplate aligned with each other. A closed cavity is formed between the first baseplate and the second baseplate. The cavity forms two channels that can implement the simultaneous detection of two sample solutions. The first baseplate includes a first base substrate, an array structure layer disposed on the first base substrate and a first hydrophobic layer disposed on the array structure layer. The array structure layer includes a drive array configured to drive the sample solution to move and an optical sensing array configured to acquire an optical signal of the sample solution. The drive array is disposed at a position corresponding to all functional areas and the communication path. The optical sensing array is disposed at a position corresponding to the luminescence detection area. The second baseplate includes a second base substrate and a second hydrophobic layer disposed on the second base substrate. The first baseplate and the second baseplate are aligned and sealed together by a sealant to form a closed cavity. Multiple functional areas and a communication path may be formed in the cavity by disposing post spacers.

The filling area 100 of each channel includes a sample filling dish 101. The sample filling dish 101 is communicated with the mixing and incubating area 200 through the communication path 500, and is configured to receive the sample solution to be detected transferred by the solution transfer device 10, and move the sample solution to the mixing and incubating area 200. The second baseplate where the sample filling dish 101 is located is provided with a filling hole to enable the solution transfer device 10 to fill the sample solution into the sample filling dish 101. The sample solution may be a blood sample.

The mixing and incubating area 200 of each channel includes a magnetic particle filling dish 201, an enzyme label filling dish 202, a magnetic particle mixing channel 203 and an enzyme label mixing channel 204. Both the magnetic particle mixing channel 203 and the enzyme label mixing channel 204 are annular channels. The magnetic particle filling dish 201 is communicated with the magnetic particle mixing channel 203. The enzyme label filling dish 202 is communicated with the enzyme label mixing channel 204. The magnetic particle mixing channel 203 is respectively communicated with the filling area 100 and the enzyme label mixing channel 204. The enzyme label mixing channel 204 is respectively communicated with the magnetic particle mixing channel 203 and the luminescence detection area 300. Corresponding filling holes are respectively provided in the second baseplate at positions where the magnetic particle filling dish 201 and the enzyme label filling dish 202 are located, so that an external device can fill a magnetic particle antibody and an enzyme labeled antibody to the magnetic particle filling dish 201 and the enzyme label filling dish 202 respectively.

The magnetic particle filling dish 201 is configured to receive the magnetic particle antibodies provided by the external device, so that the magnetic particle antibodies enters the magnetic particle mixing channel 203. Under a temperature condition provided by the temperature control device 20 (such as a constant temperature of 37° C.), the drive array of the digital microfluidic chemiluminescence detection chip drives the sample solution by the electric field to move rapidly along the annular magnetic particle mixing channel 203, so that the sample solution is mixed with the magnetic particle antibodies entering the magnetic particle mixing channel 203 by circling. Antigens in the sample solution are fully combined with the magnetic particle antibodies to form a first incubation sample solution, and the first incubation sample solution after being mixed is moved to the enzyme label mixing channel 204. The first incubation sample solution includes an antigen-magnetic particle antibody complex.

The enzyme label filling dish 202 is configured to receive an enzyme labeled antibody provided by the external device and make the enzyme labeled antibody enter the enzyme label mixing channel 204. Under the temperature condition provided by the temperature control device 20, the drive array of the digital microfluidic chemiluminescence detection chip drives the first incubation sample solution by the electric field to move rapidly along the annular enzyme label mixing channel 204, so that the first incubation sample solution is mixed with the enzyme labeled antibody by circling. The first incubation sample solution is fully combined with the enzyme labeled antibodies to form a second incubation sample solution, and the second incubation sample solution after being mixed is moved to the luminescence detection area 300. The second incubation sample solution includes an antigen-magnetic particle antibody-enzyme labeled antibody complex.

The luminescence detection area 300 of each channel includes a wash filling dish 301, a substrate filling dish 302, a purification channel 303 and a detection area 310. The purification channel 303 is an annular channel and is respectively communicated with the wash filling dish 301 and the substrate filling dish 302. In addition, the purification channel 303 is also communicated with the mixing and incubating area 200, the waste liquid area 400 and the detection area 310. Corresponding filling holes are respectively provided in the second baseplate at positions where the wash filling dish 301 and the substrate filling dish 302 are located, so that the external device can fill a wash buffer and a luminescent substrate to the wash filling dish 301 and the substrate filling dish 302 respectively.

On the one hand, the luminescence detection area 300 is configured to wash the second incubation sample solution, that is, to implement the separation of the second incubation sample solution and impurity solution. After the mixed second incubation sample is moved from the mixed incubation area 200 to the luminescence detection area 300, firstly a magnetic field is applied by the magnetic control device 30 to fix the magnetic particle antibodies in the second incubation sample to the luminescence detection area 300, and then the impurity solution is moved to the waste liquid area 400 under the driving of the drive array to complete the separation of the second incubation sample and the impurity solution. Subsequently, the magnetic field of the magnetic control device 30 is cancelled, so that the drive array can drive the second incubation sample to move. The impurity solution refers to the solution except the antigen-magnetic particle antibody-enzyme labeled antibody complex. In this embodiment, a method for fixing the magnetic particle antibodies in the second incubation sample solution may include, for example, disposing the magnetic control device 30 in an area where the purification channel 303 is located, controlling the magnetic control device 30 to be powered on, attracting the magnetic particle antibodies through the magnetic field generated by the magnetic control device 30, and adsorbing the magnetic particle antibodies on a surface in the cavity. In an appropriate magnetic field, small magnetic particle antibodies converges into a very dense magnet, and will not be taken away by the impurity solution, thus implementing the separation of the magnetic particle antibodies and the impurity solution. After the impurity solution is removed, the magnetic control device 30 is controlled to be powered off, the magnetic field disappears, and the magnetic particle antibodies can move under the electric field applied by the drive array.

The wash filling dish 301 is configured to receive the wash buffer provided by the external device and make the wash buffer enter the purification channel 303. The drive array of the digital microfluidic chemiluminescence detection chip drives the second incubation sample solution and wash buffer by the electric field to move rapidly along the annular purification channel 303, so that the second incubation sample solution is mixed with the wash buffer through circling, and unreacted free substances mixed up with the magnetic particle antibodies are released into the wash buffer. In an embodiment of the present disclosure, the above separation and washing process may be repeated for many times. The impurity solution is transported to the waste liquid area 400 by fixing the magnetic particle antibodies to complete the separation of the magnetic particle antibodies and the impurity solution, and the washing of the second incubation sample solution is completed by rapidly moving the second incubation sample solution and the wash buffer in the purification channel 303. After many times of separation and washing, a pure second incubation sample solution, namely antigen-magnetic particle antibody-enzyme labeled antibody complex, can be obtained.

On the other hand, the luminescence detection region 300 is configured to form an antigen-magnetic particle antibody-enzyme labeled antibody-luminescent substrate complex. The substrate filling dish 302 is configured to receive luminescent substrate provided by the external device and make the luminescent substrate enter the purification channel 303. The drive array of the digital microfluidic chemiluminescence detection chip drives the pure second incubation sample solution by the electric field to move rapidly along the annular purification channel 303, so that the second incubation sample solution is mixed the luminescent substrate by circling, the second incubation sample solution is fully combined with the luminescent substrate to form a third incubation sample solution, and the mixed third incubation sample solution is moved to the detection area 310. The third incubation sample solution includes an antigen-magnetic particle antibody-enzyme labeled antibody-luminescent substrate complex.

In an embodiment of the present disclosure, the detection area 310 is located in the middle of the annular purification channel 303, communicated with the purification channel 303, and is configured to implement light acquisition of chemiluminescence of the third incubation sample solution. After the mixed third incubation sample solution is moved to the detection area 310, the optical sensing array of the digital microfluidic chemiluminescence detection chip acquire an optical signal of chemiluminescence of the third incubation sample solution and converts the optical signal into an electrical signal. After that, the electrical signal is transmitted to the signal processing device 50, and the signal processing device 50 obtains concentration information through analysis and processing.

The waste liquid area 400 of each channel includes a waste liquid storage dish 401. The waste liquid storage dish 401 is communicated with the luminescence detection area 300 through the communication path 500 and is configured to receive waste liquid transferred by the luminescence detection area 300. A solution taking hole is provided in the second baseplate at a position where the waste liquid storage dish 401 is located, so as to allow the external device to take away the waste liquid.

Although description is made with the digital microfluidic chemiluminescence detection chip with the dual-channel structure as an example, embodiments of the present disclosure are also applicable to a single-channel structure or a multi-channel structure for parallel operation. In an embodiment of the present disclosure, since the mixing and incubating areas 200 of the two channels are respectively communicated with each other and the luminescence detection areas 300 of the two channels are also communicated with each other, the mixing and incubating areas 200 of the two channels may share one magnetic particle filling dish 201 and one enzyme label filling dish 202, and the luminescence detection areas 300 of the two channels may share one washing filling dish 301 and one substrate filling dish 302. In practical implementation, each channel may also be separately provided with a corresponding filling dish. In addition, the manner of implementing the mixing is not limited to circling, but may be linear oscillation, that is, controlling droplets to quickly oscillate along a linear path. Both the circling and the linear oscillation can break an equilibrium state of substances carried in the droplets and accelerate the dispersion speed of the substances in the droplets.

FIG. 4 illustrates a schematic diagram of a sample solution incubation process according to an embodiment of the present disclosure. As shown in FIG. 4, antigens in the sample solution are fully mixed with magnetic particle antibodies to form an antigen-magnetic particle antibody complex (first incubation sample solution), wherein the antigen-magnetic particle antibody complex is fully mixed with enzyme labeled antibodies to form an antigen-magnetic particle antibody-enzyme labeled antibody complex (second incubation sample solution), and the antigen-magnetic particle antibody-enzyme labeled antibody complex is fully mixed with luminescent substrate to form an antigen-magnetic particle antibody-enzyme labeled antibody-luminescent substrate complex (third incubation sample solution). In this way, the optical sensing array of the digital microfluidic chemiluminescence detection chip can implement the content detection of the sample to be detected by acquiring the optical signal generated in the chemiluminescence process of the antigen-magnetic particle antibody-enzyme labeled antibody-luminescent substrate complex.

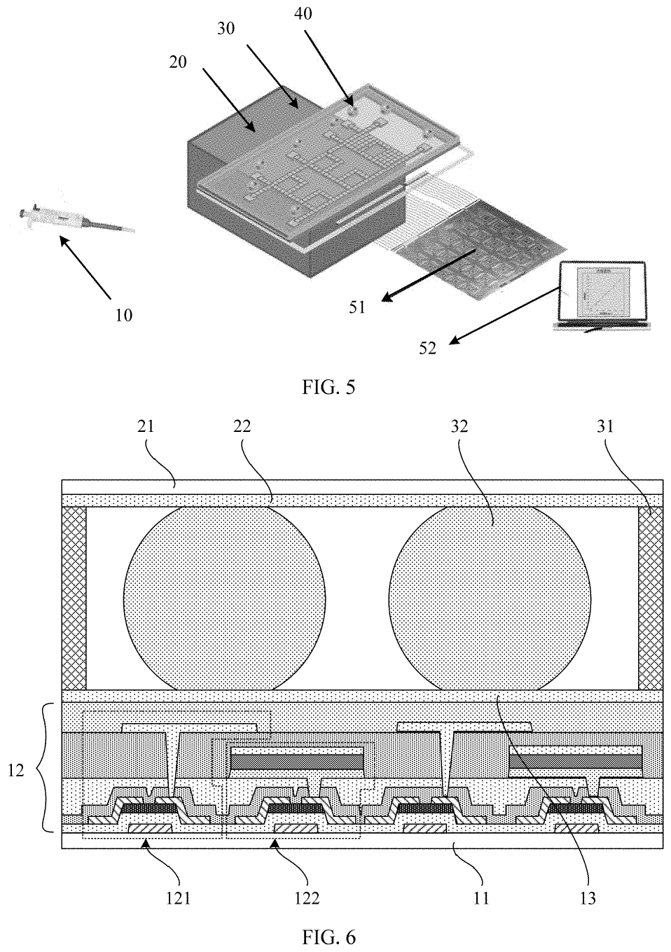

FIG. 5 illustrates a schematic diagram of a structure of a digital microfluidic chemiluminescence detection device according to an embodiment of the present disclosure. As shown in FIG. 5, in this embodiment, the solution transfer device 10 is a sample transfer gun, which is configured to transfer the sample solution to the digital microfluidic chemiluminescence detection chip 40. The temperature control device 20 and the magnetic control device 30 are a combined temperature control and magnetic control module, which is disposed on a lower side of the digital microfluidic chemiluminescence detection chip 40 and is configured to provide a set temperature and a set magnetic field for the digital microfluidic chemiluminescence detection chip 40. The signal processing device 50 includes a reading module 51 and a processing and display module 52. The reading module 51 is electrically connected with the digital microfluidic chemiluminescence detection chip 40 and is configured to read an electrical signal from the optical sensing array of the digital microfluidic chemiluminescence detection chip 40, and transmit the electrical signal to the processing and display module 52. The processing and display module 52 is connected to the reading module 51 and is configured to receive the electrical signal transmitted by the reading module 51, analyze and process the electrical signal to obtain and display concentration information. In practical implementation, the control unit may be integrated into the reading module, and the control unit implements the timing control of the drive array in the digital microfluidic chemiluminescence detection chip, the acquisition timing of the optical sensing array, the reading timing of the electrical signal and the control timing of the temperature control and magnetic control device, etc.

When the digital microfluidic chemiluminescence detection device in this embodiment is applied to detection, a single detection process mainly involves magnetic particle incubation, enzyme label incubation, washing, luminescence mixing, optical detection and other steps. Taking detection of a blood sample as an example, a process flow includes:

(1) In the mixing and incubating area of the digital microfluidic chemiluminescence detection chip, the blood sample is firstly mixed with magnetic particle antibodies by circling. Under a constant temperature condition of 37° C. of the temperature control system, antigens in the blood are fully combined with the magnetic particle antibodies to form an antigen-magnetic particle antibody complex (first incubation sample solution).

(2) In the same mixing and incubating area, the antigen-magnetic particle antibody complex and the enzyme labeled antibody are subjected to the same mixing and incubation operation to form an antigen-magnetic particle antibody-enzyme labeled antibody complex (second incubation sample solution).

(3) In the luminescence detection area of the digital microfluidic chemiluminescence detection chip, after the antigen-magnetic particle antibody-enzyme labeled antibody complex enters the luminescence detection area, first the antigen-magnetic particle antibody-enzyme labeled antibody complex is controlled by the magnetic control device to be fixed at a certain position in the luminescence detection area, impurity solution except the complex is moved to the waste liquid area by using the drive array of the digital microfluidic chemiluminescence detection chip, then wash buffer is manipulated to be mixed with the antigen-magnetic particle antibody-enzyme labeled antibody complex, circling is performed for mixing to form a suspension solution of the antigen-magnetic particle antibody-enzyme labeled antibody complex. Then the complex is fixed again, the impurity solution is moved to the waste liquid area, and after repeated washing, a pure antigen-magnetic particle antibody-enzyme labeled antibody complex can be obtained. Solution transfer is to apply a series of pre-programmed voltage sequences to the drive array of the digital microfluidic chemiluminescence detection chip, after which the droplets will move on a surface of the chip according to a predetermined path to achieve orderly work.

(4) In the same luminescence detection area, luminescent substrate is mixed with the antigen-magnetic particle antibody-enzyme labeled antibody complex by circling to form an antigen-magnetic particle antibody-enzyme labeled antibody-luminescent substrate complex (third incubation sample solution).

(5) The antigen-magnetic particle antibody-enzyme labeled antibody-luminescent substrate complex is moved to the detection area, an optical signal of chemiluminescence is acquired by the optical sensing array of the digital microfluidic chemiluminescence detection chip, and the optical signal is converted into an electrical signal. The electrical signal of the optical sensing array is read by the reading module and transmitted to a display module (such as PC). Through the analysis and processing of the electrical signal, the concentration of the detected substance is finally output.

In practical implementation, include steps such as adding samples may be further included. Adding samples is to fill a required sample, a magnetic particle antibody, an enzyme labeled antibody, a wash solution, a luminescent substrate and the like into the corresponding filling holes of the digital microfluidic chemiluminescence detection chip through the solution transfer device.

FIG. 6 is a schematic diagram of a structure of a digital microfluidic chemiluminescence detection chip integrated with an optical sensing array according to an embodiment of the present disclosure, which illustrates a sectional structure of a position where the detection area in the digital microfluidic chemiluminescence detection chip is located. The drive array adopts an active drive implementation mode, and can accurately control the movement of a separate droplet at a certain position. As shown in FIG. 6, the digital microfluidic chemiluminescence detection chip according to the embodiment of the present disclosure includes a first baseplate and a second baseplate disposed oppositely. The first baseplate includes a first base substrate 11, an array structure layer 12 disposed on a side of the first base substrate facing the second baseplate, and a first hydrophobic layer 13 disposed on a side of the array structure layer facing the second baseplate. The second baseplate includes a second base substrate 21 and a second hydrophobic layer 22 disposed on a side of the second base substrate facing the first baseplate. The first baseplate and the second baseplate are aligned and sealed together by a sealant to form a closed cavity between the first hydrophobic layer 13 of the first baseplate and the second hydrophobic layer 22 of the second baseplate. The array structure layer 12 includes a drive array 121 and an optical sensing array 122. The drive array 121 includes multiple drive units. Each drive unit is configured to drive the incubation sample solution 32 to move. Each drive unit includes a drive transistor and a drive electrode connected to the drive transistor. The optical sensing array 122 includes multiple optical sensing units. Each optical sensing unit is configured to acquire an optical signal generated in a chemiluminescence process of the incubation sample solution 32 and convert the optical signal into an electrical signal. Each optical sensing unit includes a sensing transistor and a photodiode connected with the sensing transistor.

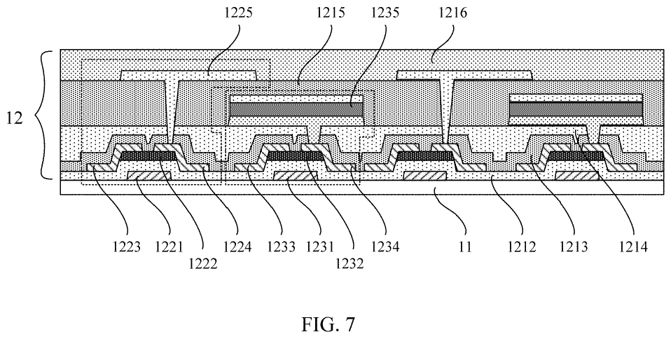

FIG. 7 illustrates a schematic diagram of a structure of an array structure layer according to an embodiment of the present disclosure. As shown in FIG. 7, the array structure layer 12 includes:

a first base substrate 11;

a drive gate electrode 1221 and a sensing gate electrode 1231 disposed on the first base substrate 11;

a first insulating layer 1212 covering the drive gate electrode 1221 and the sensing gate electrode 1231;

a drive active layer 1222 and a sensing active layer 1232 disposed on the first insulating layer 1212;

a drive source electrode 1223, a drive drain electrode 1224, a sensing source electrode 1233 and a sensing drain electrode 1234, wherein adjacent ends of the drive source electrode 1223 and the drive drain electrode 1224 are respectively disposed on the drive active layer 1222 (the end of the drive source electrode 1223 adjacent to the drive drain electrode 1224 is disposed on the drive active layer 1222, and the end of the drive drain electrode 1224 adjacent to the drive source electrode 1223 is disposed on the drive active layer 1222), and a drive channel is formed between the drive source electrode 1223 and the drive drain electrode 1224; adjacent ends of the sensing source electrode 1233 and the sensing drain electrode 1234 are respectively disposed on the sensing active layer 1232 (the end of the sensing source electrode 1233 adjacent to the sensing drain electrode 1234 is disposed on the sensing active layer 1232, and the end of the sensing drain electrode 1234 adjacent to the sensing source electrode 1233 is disposed on the sensing active layer 1232), and a sensing channel is formed between the sensing source electrode 1233 and the sensing drain electrode 1234;

a second insulating layer 1213 and a third insulating layer 1214 covering the drive source electrode 1223, the drive drain electrode 1224, the sensing source electrode 1233 and the sensing drain electrode 1234, wherein the second insulating layer 1213 and the third insulating layer 1214 are provided with a first hole exposing the sensing drain electrode 1234;

a photodiode 1235 disposed on the third insulating layer 1214, wherein a first electrode of the photodiode 1235 is connected to the sensing drain electrode 1234 through the first hole;

a fourth insulating layer 1215 covering the photodiode 1235 and provided with a second hole exposing the drive drain electrode 1224;

a drive electrode 1225 disposed on the fourth insulating layer 1215, wherein the drive electrode 1225 is connected with the drive drain electrode 1224 through the second hole; and

a fifth insulating layer 1216 covering the drive electrode 1225.

The drive gate electrode 1221, the drive active layer 1222, the drive source electrode 1223 and the drive drain electrode 1224 form a drive transistor. The drive transistor and the drive electrode 1225 form a drive unit. The sensing gate electrode 1231, the sensing active layer 1232, the sensing source electrode 1233 and the sensing drain electrode 1234 form a sensing transistor. The sensing transistor and the photodiode 1235 form an optical sensing unit. In this way, the drive unit and the optical sensing unit can be formed on the first base substrate 11 through the same manufacturing process. In an embodiment of the present disclosure, the photodiode 1235 may be a PIN-type photodiode, which includes a P-type semiconductor layer, an N-type semiconductor layer and an intrinsic semiconductor layer disposed between the P-type semiconductor layer and the N-type semiconductor layer.

A process of manufacturing the array structure layer according to the embodiment of the present disclosure may include:

(1) A first metal thin film is deposited on a base substrate 11, and the first metal thin film is patterned through a patterning process to form patterns of a drive gate electrode 1221 and a sensing gate electrode 1231.

(2) A first insulating thin film and an active layer thin film are sequentially deposited on the base substrate on which the above structure is formed, and the active layer thin film is patterned through a patterning process to form patterns of a first insulating layer 1212 covering the base substrate 11 and a drive active layer 1222 and a sensing active layer 1232 disposed on the first insulating layer 1212.

(3) A second metal thin film is deposited on the substrate on which the above structure is formed, and the second metal thin film is patterned through a patterning process to form patterns of a drive source electrode 1223, a drive drain electrode 1224, a sensing source electrode 1233 and a sensing drain electrode 1234. Adjacent ends of the drive source electrode 1223 and the drive drain electrode 1224 are respectively disposed on the drive active layer 1222. A drive channel is formed between the drive source electrode 1223 and the drive drain electrode 1224. Ends of the drive source electrode 1223 and the drive drain electrode 1224 away from each other are respectively disposed on the first insulating layer 1212 (which includes that the end of the drive source electrode 1223 away from the drive drain electrode 1224 is disposed on the first insulating layer 1212, and the end of the drive drain electrode 1224 away from the drive source electrode 1223 is disposed on the first insulating layer 1212). Adjacent ends of the sensing source electrode 1233 and the sensing drain electrode 1234 are respectively disposed on the sensing active layer 1232. A sensing channel is formed between the sensing source electrode 1233 and the sensing drain electrode 1234. Ends of the sensing source electrode 1233 and the sensing drain electrode 1234 away from each other are respectively disposed on the first insulating layer 1212 (which includes that the end of the sensing source electrode 1233 away from the sensing drain electrode 1234 is disposed on the first insulating layer 1212, and the end of the sensing drain electrode 1234 away from the sensing source electrode 1233 is disposed on the first insulating layer 1212).

(4) Firstly a second insulating thin film is deposited on the base substrate on which the above structure is formed, then a third insulating thin film is coated, and the second insulating thin film and the third insulating thin film are patterned through a patterning process to form a second insulating layer 1213 and a third insulating layer 1214 covering the base substrate 11, which are provided with a first hole exposing the sensing drain electrode 1234.

(5) A P-type semiconductor layer, an intrinsic semiconductor layer and an N-type semiconductor layer are sequentially deposited on the base substrate on which the above structure is formed, a pattern of a photodiode 1235 is formed through a patterning process, and the P-type semiconductor layer of the photodiode 1235 is connected to the sensing drain electrode 1234 through the first hole.

(6) A fourth insulating thin film is coated on the base substrate on which the above structure is formed, and the fourth insulating thin film is patterned through a patterning process to form a fourth insulating layer 1215 covering the photodiode 1235, which is provided with a second hole exposing the drive drain electrode 1224.

(7) A transparent conducting thin film is deposited on the base substrate on which the above structure is formed, the transparent conducting thin film is patterned through a patterning process, and a pattern of a drive electrode 1225 is formed on the fourth insulating layer 1215.

(8) A fifth insulating thin film is coated on the base substrate on which the above structure is formed to form a fifth insulating layer 1216 covering the drive electrode 1225.

The first insulating layer and the second insulating layer may be made of silicon oxide (SiOx), silicon nitride (SiNx) or silicon oxynitride (SiON), and may be in a single-layer structure or a multi-layer composite structure. The first insulating layer is called gate insulating (GI) layer and the second insulating layer is called interlayer dielectric (ILD) layer. The third insulating layer, the fourth insulating layer and the fifth insulating layer may be made of an organic material, and are called planarization (PLN) layers. The first metal thin film and the second metal thin film may be made of a metal material, such as silver (Ag), copper (Cu), aluminum (Al) or molybdenum (Mo), or an alloy material consisting of the above metals, and may be in a single-layer structure or a multi-layer composite structure. The transparent conducting thin film may be made of indium tin oxide (ITO) or indium zinc oxide (IZO). The active layer thin film may be made of amorphous indium gallium zinc oxide (a-IGZO), zinc oxynitride (ZnON), indium zinc tin oxide (IZTO), amorphous silicon (a-Si), polysilicon (p-Si), hexathiophene or polythiophene, that is, the embodiments of the present disclosure are applicable to thin film transistors manufactured based on oxide technology, silicon technology or organic technology.

It should be noted that the above structure and its manufacturing process are only exemplary. In an exemplary embodiment, the corresponding structure may be changed and the patterning processes may be added or removed according to the actual needs. For example, a thin film transistor may have a top gate structure, a bottom gate structure, a single gate structure, or a double gate structure. Other electrodes, leads and structural film layers may also be disposed in the array structure layer, which are not specifically limited in the embodiments of the present disclosure.

The embodiments of the present disclosure provide a digital microfluidic chemiluminescence detection chip and a detection device, which use the digital microfluidic technology to implement preparation of the complex sample solution of chemiluminescence reaction, avoid the complex fluid circuit system, implement an automatic and high-accuracy pretreatment of the sample solution to be detected, can ensure an accurate ratio of the sample solution to reagent, ensure the repeatability and stability of experimental results, have features such as compact structure, small volume, low power consumption and low cost, and can implement the rapid and accurate POCT detection of trace substances. By integrating the optical sensor array in the digital microfluidic chemiluminescence detection chip, the acquisition of the optical signal in the chip is implemented, the complex peripheral optical path structure is avoided, the signal-to-noise ratio of the signal is reduced, and the consistency of detection results is improved. The embodiment of the present disclosure minimizes the system volume to a greatest extent, improves the consistency of detection results, effectively solves the defects of large system volume and low consistency of detection results of the existing chemiluminescence detection devices, and has a wide application prospect.

Based on the technical concept of the embodiment of the present disclosure, an embodiment of the present disclosure further provides a method for detecting digital microfluidic chemiluminescence using the digital microfluidic chemiluminescence detection chip. The method for detecting digital microfluidic chemiluminescence according to the embodiment of the present disclosure includes the following steps:

In step S1, the drive array drives the sample solution to be sequentially combined with the magnetic particle antibody, the enzyme labeled antibody and the luminescent substrate to form an antigen-magnetic particle antibody-enzyme labeled antibody-luminescent substrate complex.

In step S2, the optical sensing array acquires an optical signal of chemiluminescence of the antigen-magnetic particle antibody-enzyme labeled antibody-luminescent substrate complex, and converts the optical signal into an electrical signal.

Step S1 includes the following steps: In step S11, in the mixing and incubating area, the drive array drives the sample solution to be sequentially combined with the magnetic particle antibody and the enzyme labeled antibody to form an antigen-magnetic particle antibody-enzyme labeled antibody complex.

In step S12, in the luminescence detection area, the drive array drives the antigen-magnetic particle antibody-enzyme labeled antibody complex to be combined with luminescent substrate to form an antigen-magnetic particle antibody-enzyme label ed antibody-luminescent substrate complex.

Step S11 includes the following steps:

In the magnetic particle mixing channel in the mixing and incubating area, the drive array drives the sample solution to be combined with the magnetic particle antibody to form an antigen-magnetic particle antibody complex.

In the enzyme label mixing channel in the mixing and incubating area, the drive array drives the antigen-magnetic particle antibody complex to be combined with the enzyme labeled antibody to form an antigen-magnetic particle antibody-enzyme labeled antibody complex.

Step S12 includes the following steps:

In the purification channel of the luminescence detection area, the drive array drives the antigen-magnetic particle antibody-enzyme labeled antibody complex to be mixed with a wash buffer and discharges an impurity solution from the purification channel.

In the purification channel of the luminescence detection area, the drive array drives the antigen-magnetic particle antibody-enzyme labeled antibody complex to be combined with a luminescent substrate to form an antigen-magnetic particle antibody-enzyme labeled antibody-luminescent substrate complex.

Step S2 includes the following steps:

In the detection area of the luminescence detection area, the optical sensing array acquires an optical signal of chemiluminescence of the antigen-magnetic particle antibody-enzyme labeled antibody-luminescent substrate complex, and converts the optical signal into an electrical signal.

The method for detecting digital microfluidic chemiluminescence provided by the embodiment of the present disclosure uses the digital microfluidic technology to implement the preparation of the complex sample solution of chemiluminescence reaction, avoids the complex fluid path system, and avoids the complex peripheral optical path structure by acquiring the optical signal in the digital microfluidic chemiluminescence detection chip. The method provided by the embodiment of the present disclosure reduces the signal-to-noise ratio of the signal and improves the consistency of detection results.

In the description of the embodiment of the present application, it should be understood that the orientation or positional relations indicated by terms such as “middle”, “up”, “down”, “front”, “back”, “vertical”, “horizontal”, “top”, “bottom”, “inside” and “outside” are the orientation or positional relations based on the drawings, only for the convenience of describing the present disclosure and simplifying the description, instead of indicating or implying that the device or element referred to must have a specific orientation or be constructed and operated in a specific orientation, so they should not be understood as limitations on the present disclosure.

In the description of the embodiment of the present disclosure, it should be noted that unless otherwise specified and limited, the terms “mount”, “connected” and “connect” should be understood in a broad sense. For example, a connection may be fixed connection, detachable connection or integrated connection, may be mechanical connection or electrical connection, or may be direct connection, indirect connection through intermediate medium, or communication inside two components. For those skilled in the art, the specific meanings of the above terms in the present disclosure can be understood according to the actual situation.

Although the embodiments disclosed in the present disclosure are as above, the contents described are only embodiments adopted for the convenience of understanding the present disclosure and are not used to limit the present disclosure. Any person skilled in the art to which the present disclosure pertains may make any modification and variation in the form and details of implementation without departing from the essence and scope disclosed in the present disclosure. However, the scope of protection of the present disclosure shall still be subject to the scope defined by the appended claims.

What is claimed is:

1. A digital microfluidic chemiluminescence detection chip, comprising a first baseplate and a second baseplate disposed oppositely, wherein a cavity formed by the first baseplate and the second baseplate comprises a mixing and incubating area configured to implement combination of an antigen, a magnetic particle antibody and an antibody, a luminescence detection area configured to implement chemiluminescence and detect an optical signal, and a communication path configured to communicate the mixing and incubating area with the luminescence detection area, the first baseplate is provided with a drive array configured to drive a sample solution to move and an optical sensing array configured to acquire a luminescence signal of the sample solution, the drive array corresponds to positions of the mixing and incubating area, the luminescence detection area and the communication path, and the optical sensing array corresponds to a position of the luminescence detection area.

2. The digital microfluidic chemiluminescence detection chip according to claim 1, wherein the mixing and incubating area comprises a magnetic particle filling dish and a magnetic particle mixing channel, the magnetic particle filling dish is configured to provide the magnetic particle antibody to the magnetic particle mixing channel, in the magnetic particle mixing channel, the sample solution moves along the magnetic particle mixing channel under drive of the drive array to make the antigen in the sample solution be combined with the magnetic particle antibody to form a first incubation sample solution, and the first incubation sample solution comprises an antigen-magnetic particle antibody complex.

3. The digital microfluidic chemiluminescence detection chip according to claim 2, wherein the mixing and incubating area further comprises an enzyme label filling dish and an enzyme label mixing channel, the enzyme label filling dish is configured to provide an enzyme labeled antibody to the enzyme label mixing channel, the enzyme label mixing channel is communicated with the magnetic particle mixing channel, in the enzyme label mixing channel, the first incubation sample moves along the enzyme label mixing channel under the drive of the drive array to make the first incubation sample solution be combined with the enzyme labeled antibody to form a second incubation sample solution, and the second incubation sample solution comprises an antigen-magnetic particle antibody-enzyme labeled antibody complex.

4. The digital microfluidic chemiluminescence detection chip according to claim 3, wherein the luminescence detection area comprises a substrate filling dish and a purification channel, the purification channel is communicated with the mixing and incubating area, the substrate filling dish is configured to provide a luminescent substrate to the purification channel, in the purification channel, the second incubation sample moves along the purification channel under the drive of the drive array to make the second incubation sample be combined with the luminescent substrate to form a third incubation sample solution, and the third incubation sample solution comprises an antigen-magnetic particle antibody-enzyme labeled antibody-luminescent substrate complex.

5. The digital microfluidic chemiluminescence detection chip according to claim 4, wherein the magnetic particle mixing channel, the enzyme label mixing channel and the purification channel are annular channels, and mixing of the sample solution and the magnetic particle antibody, mixing of the first incubation sample solution and the enzyme labeled antibody, and mixing of the second incubation sample solution and the luminescent substrate are implemented by circling.

6. The digital microfluidic chemiluminescence detection chip according to claim 4, wherein the luminescence detection area further comprises a wash filling dish, the wash filling dish is configured to provide a wash buffer to the purification channel, and in the purification channel, the second incubation sample solution moves along the purification channel under the drive of the drive array to implement mixing of the second incubation sample solution and the wash buffer; an external magnetic control device fixes the magnetic particle antibody in the second incubation sample solution, and an impurity solution in the second incubation sample solution is discharged from the purification channel under the drive of the drive array.

7. The digital microfluidic chemiluminescence detection chip according to claim 4, wherein the luminescence detection area further comprises a detection area, the detection area is communicated with the purification channel, and in the detection area, the optical sensing array acquires an optical signal of chemiluminescence of the third incubation sample solution and converts the optical signal into an electrical signal.

8. The digital microfluidic chemiluminescence detection chip according to claim 6, wherein the second baseplate is provided with a plurality of filling holes, and the plurality of filling holes correspond to positions of the magnetic particle filling dish, the enzyme label filling dish, the wash filling dish and the substrate filling dish respectively.

9. The digital microfluidic chemiluminescence detection chip according to claim 1, wherein the digital microfluidic chemiluminescence detection chip further comprises a filling area and a waste liquid area, the filling area is communicated with the mixing and incubating area and is configured to receive a sample solution to be detected, and the waste liquid area is communicated with the luminescence detection area and is configured to receive a waste liquid from the luminescence detection area.

10. The digital microfluidic chemiluminescence detection chip according to claim 1, wherein the drive array adopts an active drive implementation mode.

11. The digital microfluidic chemiluminescence detection chip according to claim 1, wherein the first baseplate comprises a first base substrate, an array structure layer disposed on a side of the first base substrate facing the second baseplate and a first hydrophobic layer disposed on a side of the array structure layer facing the second baseplate, the second baseplate comprises a second base substrate and a second hydrophobic layer disposed on a side of the second base substrate facing the first baseplate, the drive array and the optical sensing array are disposed in the array structure layer, the drive array comprises a plurality of drive units, each drive unit comprises a drive transistor and a drive electrode, the drive electrode is connected to the drive transistor, the optical sensing array comprises a plurality of optical sensing units, each optical sensing unit comprises a sensing transistor and a photodiode, and the photodiode is connected to the sensing transistor.

12. The digital microfluidic chemiluminescence detection chip according to claim 11, wherein the array structure layer comprises:
a first base substrate;
a drive gate electrode and a sensing gate electrode disposed on the first base substrate;
a first insulating layer covering the drive gate electrode and the sensing gate electrode;
a drive active layer and a sensing active layer disposed on the first insulating layer;
a drive source electrode and a drive drain electrode with adjacent ends respectively disposed on the drive active layer, and a sensing source electrode and a sensing drain electrode with adjacent ends disposed on the sensing active layer;
a second insulating layer and a third insulating layer covering the drive source electrode, the drive drain electrode, the sensing source electrode and the sensing drain electrode, and provided with a first hole exposing the sensing drain electrode;
a photodiode disposed on the third insulating layer, wherein a first electrode of the photodiode is connected with the sensing drain electrode through the first hole;
a fourth insulating layer covering the photodiode and provided with a second hole exposing the drive drain electrode;
a drive electrode disposed on the fourth insulating layer, wherein the drive electrode is connected with the drive drain electrode through the second hole; and
a fifth insulating layer covering the drive electrode.

13. A digital microfluidic chemiluminescence detection device, comprising the digital microfluidic chemiluminescence detection chip according to claim 1, and further comprising a solution transfer device, a temperature control device, a magnetic control device and a signal processing device, wherein the solution transfer device is configured to transfer a sample solution onto the digital microfluidic chemiluminescence detection chip, the temperature control device is configured to provide a set temperature to the digital microfluidic chemiluminescence detection chip, the magnetic control device is configured to provide a set magnetic field to the digital microfluidic chemiluminescence detection chip, and the signal processing device is connected with the digital microfluidic chemiluminescence detection chip and is configured to read an electrical signal of the optical sensing array, and analyze and process the electrical signal to obtain concentration information.

14. The digital microfluidic chemiluminescence detection device according to claim 13, wherein the temperature control device is disposed on a side of the first baseplate away from the second baseplate or a side of the second baseplate away from the first baseplate and is configured to provide the set temperature to the mixing and incubating area; the magnetic control device is disposed on the side of the first baseplate away from the second baseplate or the side of the second baseplate away from the first baseplate and is configured to provide the set magnetic field to the luminescence detection area.

15. A method for detecting digital microfluidic chemiluminescence using the digital microfluidic chemiluminescence detection chip according to claim 1, comprising:
driving, by the drive array, a sample solution to be sequentially combined with the magnetic particle antibody, the enzyme labeled antibody and the luminescent substrate to form an antigen-magnetic particle antibody-enzyme labeled antibody-luminescent substrate complex; and
acquiring, by the optical sensing array, an optical signal of chemiluminescence of the antigen-magnetic particle antibody-enzyme labeled antibody-luminescent substrate complex, and converting the optical signal into an electrical signal.

16. The method for detecting digital microfluidic chemiluminescence according to claim 15, wherein driving, by the drive array, the sample solution to be sequentially combined with the magnetic particle antibody, the enzyme labeled antibody and the luminescent substrate to form the antigen-magnetic particle antibody-enzyme labeled antibody-luminescent substrate complex comprises:
driving, by the drive array, the sample solution to be sequentially combined with the magnetic particle antibody and the enzyme labeled antibody in the mixing and incubating area to form an antigen-magnetic particle antibody-enzyme labeled antibody complex; and
driving, by the drive array, the antigen-magnetic particle antibody-enzyme labeled antibody complex to be combined with the luminescent substrate in the luminescence detection area to form the antigen-magnetic particle antibody-enzyme labeled antibody-luminescent substrate complex.

17. The digital microfluidic chemiluminescence detection chip according to claim 2, wherein the drive array adopts an active drive implementation mode.

18. The digital microfluidic chemiluminescence detection chip according to claim 3, wherein the drive array adopts an active drive implementation mode.

19. The digital microfluidic chemiluminescence detection chip according to claim 4, wherein the drive array adopts an active drive implementation mode.

20. The digital microfluidic chemiluminescence detection chip according to claim 5, wherein the drive array adopts an active drive implementation mode.
